(19) United States
(12) Patent Application Publication
(10) Pub. No.: US 2016/0045586 A1
(43) Pub. Date: Feb. 18, 2016

(54) Toxoid, Compositions and Related Methods

(71) Applicant: SANOFI PASTEUR, INC., Swiftwater, PA (US)

(72) Inventor: Steven Hauser, Swiftwater, PA (US)

(73) Assignee: Sanofi Pasteur, Inc., Swiftwater, PA (US)

(21) Appl. No.: 14/776,145

(22) PCT Filed: Mar. 14, 2014
(86) PCT No.: PCT/US14/29035

§ 371 Date: Sep. 14, 2015

Related U.S. Application Data
(60) Provisional application No. 61/790,423, filed on Mar. 15, 2013.

(51) Int. Cl. A61K 39/08; C07K 14/33

(52) U.S. Cl. CPC A61K 39/08; C07K 14/33

(57) ABSTRACT

The disclosure relates to generally to the field of toxin inactivation. More specifically, it relates to clostridial toxins, methods of inactivating these toxins and compositions (e.g., vaccines) comprising toxoids (e.g., produced by these methods). Provided are methods of producing a C. difficile toxoid comprising inactivating a C. difficile toxin with formaldehyde. Toxoids prepared by these methods are stable at high temperature (e.g., 37° C.) and remain non-cytotoxic with minimal residual formaldehyde.

### RELATED APPLICATIONS

This application claims priority to U.S. Ser. No. 61/790,423 filed Mar. 15, 2013 which is hereby incorporated in its entirety into this application.

### FIELD OF THE DISCLOSURE

The disclosure relates generally to the field of toxin inactivation. More specifically, it relates to clostridial toxins, methods of inactivating these toxins and compositions (e.g., vaccines) comprising the resulting toxoids.

### BACKGROUND OF THE DISCLOSURE

Bacterial toxins may be inactivated using chemical agents well known to those of skill in the art such as, for example, formaldehyde, glutaraldehyde or B-priopiolactone. Inactivated toxins (also known as toxoids) may in some circumstances revert or regain cytotoxicity.

One Clostridium difficile vaccine is a formalin-inactivated vaccine that contains toxoids A and B purified from anaerobic cultures of Clostridium difficile strain ATCC 43255. The toxins may be individually purified, inactivated (toxoided), and mixed at a targeted toxoid A: toxoid B ratio (e.g., 3:2). Formalin-mediated toxoiding of toxins A and B plays a central role in defining and controlling many of the product characteristics and quality attributes of the drug product and most importantly, the safety of the vaccine by preventing cytotoxicity.

Methods for inactivating C. difficile toxins A and B using formaldehyde have been published. For example, U.S. Pat. No. 6,669,520 describes a mixture of partially purified C. difficile toxins A and B incubated with 4.25% mg/ml formaldehyde at 4° C. for 18 days. The resulting toxoid mixture was used to prepare formulations with and without formaldehyde. In the absence of residual formalin, partial reversion to a toxic form occurred at higher temperatures (28-37° C.), with the toxoid regaining detectable biological activity over days to weeks. While residual formaldehyde may be used to prevent reversion, limiting the amount present in a vaccine is desired (e.g., to meet requirements set by some regulatory agencies). There is a need in the art for toxoids that retain stability at high temperatures (e.g., 37° C.) and contain minimal residual formaldehyde, especially to meet requirements set by various drug regulatory agencies. The methods described herein provide toxoids that are stable at high temperatures and contain only residual formalin. Such methods and additional advantages thereof are provided by this disclosure.

### BRIEF DESCRIPTION OF THE DRAWINGS

FIG. 1 is a graphical representation of the results of a cytotoxicity assay. A cytotoxicity assay using IMR90 cells was conducted using samples from one batch of each of toxin A and toxin B that underwent inactivation in accordance to the described methods (Example 2). Samples were taken on day 0, following addition of formaldehyde to inactivate the toxin and on a number of days later to assess the cytotoxicity of the material. The y-axis identifies the minimum concentration at which 50% of the cells became rounded (as opposed to their normal striated morphology) in the presence of toxic material (MC50). The lower limit of detection value (LOD) for the assay is identified using a dashed line.

FIG. 2 is a schematic representation of an exemplary method of inactivating C. difficile Toxin A and Toxin B.

FIG. 3 is a graphical representation of the results from an immunization study. In the study (described in Example 2) conducted in hamster challenge model (using 5 groups with 9 hamsters/group), Toxoid A and Toxoid B were prepared in accordance to the described methods, combined and formulated as a lyophilized composition. The composition was reconstituted and adjuvanted prior to vaccination. One hamster group was administered a placebo. Four different dilutions of a human dose (HD) of the composition (100 μg/dose) were prepared, one for each of the four other hamster groups. Compositions administered (i.e., placebo or HD dilution) are identified on X-axis. The % survival of each group (Y-axis) following administration of a lethal challenge dose of C. difficile was determined as is graphically shown.

### SUMMARY OF THE DISCLOSURE

This disclosure provides methods and reagents for preparing toxoids that are stable at high temperatures and contain only minimal formalin (e.g., residual formaldehyde). Exemplary methods produce toxoid compositions that are stable at high temperature (e.g., 37° C.) and contain low amounts (e.g., residual amounts) of formaldehyde by, among other steps, inactivating the purified Toxin A and the purified Toxin B by incubation with about any of 0.15% to about 0.5% formaldehyde (w/v) (e.g., about any of 0.2% to 0.8%, such as about 0.2% for Toxoid A (e.g., 0.21%) and/or about 0.4% (e.g., 0.42%) for Toxoid B) at an appropriate temperature (e.g., about any of 17 to 32° C. (e.g., about 25° C.)) for an appropriate amount of time (e.g., about two to about 30 days) (e.g., such that the respective toxin is inactivated into the corresponding toxoid). The toxoids may then be combined to produce a toxoid-containing immunological composition and/or vaccine that contains only a residual amount of formaldehyde (e.g., about any of 0.0001% to 0.025% such as 0.004%, 0.008%, or 0.016% (w/v)). The toxoid immunological composition may be in lyophilized form which may contain, for example, a higher concentration of formaldehyde (e.g., about 0.016% formaldehyde (w/v) than a composition reconstituted therefrom (e.g., about any of 0.001%, 0.004% or 0.008% formaldehyde (w/v)) for administration to a host. This disclosure provides methods for producing toxoids and compositions comprising such toxoids including immunological compositions and/or vaccines, as well as intermediates thereof (e.g., compositions comprising toxoid A or toxoid B alone). Other embodiments are provided in this disclosure, as will be apparent to one of ordinary skill in the art.

### DETAILED DESCRIPTION

This disclosure provides methods for preparing clostridial toxoids, clostridial toxoids prepared by these methods and compositions comprising these toxoids. Of particular interest herein are C. difficile Toxins A and/or B and/or derivatives thereof (e.g. genetically detoxified versions, truncated forms, fragments, and the like). For the purposes of this disclosure, Toxin A and/or Toxin B may include any C. difficile toxin that may be identified as Toxin A and/or Toxin B using standard techniques in the art. Exemplary techniques may include, for instance, immunoassays such as ELISA, dot blot or in vivo assays. Reagents useful in making such identifications may include, for instance, anti-Toxin A rabbit polyclonal antisera (e.g., Abcam® Product No. ab35021 or Abcam® Product No. ab93318) or an anti-Toxin A mouse monoclonal antibody (e.g., any of Abcam® Product Nos. ab19953 (mAb PCG4) or ab82285 (mAb B618M)), anti-Toxin B rabbit polyclonal antisera (e.g., Abcam® Product No. ab83066) or an anti-Toxin B mouse monoclonal antibody (e.g., any of Abcam® Product Nos. ab77583 (mAb B428M), ab130855 (mAb B423M), or ab130858 (mAb B424M)) (all available from Abcam® (Cambridge, Mass.)).

This disclosure provides methods for preparing clostridial toxoids, clostridial toxoids prepared by these methods and compositions comprising these toxoids. Of particular interest herein are C. difficile Toxins A and/or B and/or derivatives thereof (e.g. genetically detoxified versions, truncated forms, fragments, and the like). For the purposes of this disclosure, Toxin A and/or Toxin B may include any C. difficile toxin that may be identified as Toxin A and/or Toxin B using standard techniques in the art. Exemplary techniques may include, for instance, immunoassays such as ELISA, dot blot or in vivo assays. Reagents useful in making such identifications may include, for instance, anti-Toxin A rabbit polyclonal antisera (e.g., Abcam® Product No. ab35021 or Abcam® Product No. ab93318) or an anti-Toxin A mouse monoclonal antibody (e.g., any of Abcam® Product Nos. ab19953 (mAb PCG4) or ab82285 (mAb B618M)), anti-Toxin B rabbit polyclonal antisera (e.g., Abcam® Product No. ab83066) or an anti-Toxin B mouse monoclonal antibody (e.g., any of Abcam® Product Nos. ab77583 (mAb B428M), ab130855 (mAb B423M), or ab130858 (mAb B424M)) (all available from Abcam® (Cambridge, Mass.)).

Provided herein are methods for producing a C. difficile toxoid composition that is stable at high temperature (e.g., 37° C.) and contains low amounts of formaldehyde by one or more of the steps of: 1) providing a C. difficile culture comprising Toxin A and Toxin B; 2) purifying Toxin A and Toxin B from the culture to provide separate compositions of each toxin; 3) inactivating the purified Toxin A and the purified Toxin B by incubation with about any of 0.15% to about 0.5% formaldehyde (w/v) (e.g., about any of 0.2% to 0.8%, such as about 0.2% (e.g., 0.21%) for Toxoid A and/or about 0.4% (e.g., about 0.42%) for Toxoid B) at an appropriate temperature (e.g., about any of 17 to 32° C. (e.g., about 25° C.)) for an appropriate amount of time (e.g., about two to about 21 days) (e.g., such that the respective toxin is inactivated into the corresponding toxoid) to generate Toxoid A and Toxoid B compositions, respectively; and, 4) combining the toxoids to produce a toxoid immunological composition and/or vaccine that contains only a residual amount of formaldehyde (e.g., about any of 0.0001% to 0.025%, such as about any of 0.001%, 0.002%, 0.003%, 0.004%, 0.005%, 0.006%, 0.007%, 0.008%, 0.01%, 0.016%, 0.02% or 0.025% (w/v) (preferably about either of 0.004% or 0.008%)). While the amount of formaldehyde contained in the compositions is typically referred to in terms of a percentage of the composition (weight/volume (“w/v”)), it may be important to adjust the stoichiometry based on certain factors such as protein concentration. For instance, a suitable concentration of formaldehyde as contemplated herein is one that will provide intermolecular crosslinks within individual Toxin A and/or Toxin B polypeptides without also substantially crosslinking the polypeptides to one another (e.g., without producing intermolecular crosslinks). As shown in the Examples, a composition comprising 0.5 mg/ml Toxin A may only require 0.21% (w/v) formaldehyde. However, composition comprising a higher concentration of Toxin A may require a higher or lower concentration of formaldehyde to produce the required intramolecular crosslinks (e.g., toxoiding) without also producing a substantial amount of intermolecular crosslinks. The same principle may apply to the toxoiding of Toxin B. Suitable conditions for a particular composition may be determined by one of ordinary skill in the art using the techniques described herein or as may be available in the art. For instance, whether a particular amount of formaldehyde is effective for toxoiding a particular toxin in a composition may be determined using any one or more of the cytotoxicity assays, anion exchange chromatography, size exclusion chromatography, amine content analysis, antigenicity and immunogenicity assays described in the Examples section. It should also be understood that while formaldehyde is used herein, other similar agents may be substituted therefor as may be determined by one of ordinary skill in the art. For instance, in some embodiments, formaldehyde may be substituted by glutaraldehyde. Additionally, it should also be understood that while the toxoiding in phosphate buffer is used herein other similar agents may be substituted therefor as may be determined by one of ordinary skill in the art. For instance, in some embodiments, buffers containing glycine and/or lysine. While different concentrations may be required to make such a substitution, suitable conditions for such a substitution may be determined using the techniques described herein (e.g., any one or more of the cytotoxicity assays, anion exchange chromatography, size exclusion chromatography, amine content analysis, antigenicity and immunogenicity assays described in the Examples section).

In certain embodiments, Toxin A may be mixed for an appropriate amount of time (e.g., about any of one to 60 minutes, such as ten minutes) with an appropriate amount of formaldehyde (e.g., about 0.2%) formaldehyde to produce Toxoid A and then incubated at an appropriate temperature (e.g., about 25° C.) for an appropriate amount of time (e.g., about two to 21 days, such as any of about six to 12 days (e.g., about six days)). In some preferred embodiments, as shown in the Examples herein, Toxin A may be converted to Toxoid A by incubating Toxin A in a formulation comprising about 0.21% (w/v) formaldehyde at about 25° C. for about six to about 12 days. In certain embodiments, Toxin B may be mixed for an appropriate amount of time (e.g., about any of one to 60 minutes, such as ten minutes) with an appropriate amount of formaldehyde (e.g., about 0.42%) and then incubated at an appropriate temperature (e.g., about 25° C.) for an appropriate amount of time (e.g., about two to 30 days, such as any of about 13-21 days (e.g., about 21 days)) to produce Toxoid B. In some preferred embodiments, as shown in the Examples herein, Toxin B may be converted to Toxoid B by incubating mixing Toxin B in a formulation comprising about 0.42% (w/v) formaldehyde at about 25° C. for about 13 to about 20 days. The formaldehyde may be introduced (e.g., aseptically) to a desired amount into a solution comprising Toxin A or Toxin B from a stock solution of 37% formaldehyde, followed by incubation for a period of time (e.g., five to ten minutes) and storage for an appropriate temperature and time (e.g., 2-8° C. for multiple days). In certain embodiments, purified Toxin A and purified Toxin B may be combined and then mixed for an appropriate amount of time (e.g., about any of one to 60 minutes, such as ten minutes) with an appropriate amount of formaldehyde (e.g., about 0.42%) and then incubated at an appropriate temperature (e.g., about 25° C.) for an appropriate amount of time (e.g., about two to 30 days, such as any of about 13-21 days (e.g., about 21 days) to produce Toxoids A and B. The toxoids may be contained in a suitable buffer (e.g., about any of 20-150 mM phosphate (e.g., 100 mM), pH 7.0). The Toxoid A and Toxoid B compositions may then be combined in a suitable buffer (e.g., by diafiltration into an appropriate buffer such as 20 mM citrate, pH 7.5, 5%-8% sucrose (e.g., 8%)) to produce a Toxoid NB immunological composition and/or vaccine (e.g., which may be collectively referred to herein as “composition”). Such compositions may also be prepared in lypholized form using standard techniques. Thus, in some embodiments, the toxoid immunological composition may be in lyophilized form which may contain, for example, a higher concentration of formaldehyde than a composition reconstituted therefrom (e.g., the drug product). For instance, the lyophilized composition may comprise about 0.016% formaldehyde (w/v) but after reconstitution for administration to a host, the composition (e.g., drug product) may comprise less than 0.016% formaldehyde (w/v) (e.g., about any of 0.001%, 0.002%, 0.003%, 0.004%, 0.005%, 0.006%, 0.007%, 0.008%, 0.01 (w/v)). In some embodiments, then, the Toxoid A/B immunological composition and/or vaccine (e.g., “drug product”) may comprise about any of 0.0001% to 0.025% formaldehyde (w/v) (e.g., about any of 0.001%, 0.002%, 0.004%, 0.005%, 0.006%, 0.007% 0.008%, 0.01%, 0.016%, 0.02% or 0.025% (w/v)) (e.g., “residual formaldehyde”). The inclusion of residual formaldehyde in the drug product has been found to be especially beneficial in that it may reduce and/or prevent reversion of Toxoid A and/or Toxoid B to Toxin A or Toxin B, respectively, where the composition is maintained at higher temperature (e.g., above 4° C. such as room temperature or 37° C., for instance) for a period of time (e.g., about six weeks). It is noted that, in some instances, the amount of formaldehyde may be increased to reduce toxin inactivation time. The final composition (e.g., the immunological composition, vaccine) will include only a residual amount of formaldehyde. As shown in the Examples, these processes surprisingly provide immunological Toxoid A/B-containing compositions having favorable biochemical and functional properties.

In certain embodiments, it may be beneficial to, at any point in the methods described herein, regulate the amount of certain buffer components that may interfere with the functionality of formaldehyde therein. For instance, TRIS has an amine group that can effectively compete with the protein for formaldehyde mediated modification, thereby lowering the effective formaldehyde concentration in the reaction mixture. It may therefore be beneficial to maintain the amounts of TRIS in compositions in which toxins and/or toxoids are produced at a low level. For instance, the residual TRIS values in the toxin preparations may be lowered to more suitable levels (e.g., below about 1 to about 5 μg/ml (e.g., 1 μg/ml (e.g., below limit of detection) or 5 μg/ml). As shown in the Examples, the residual TRIS values in the toxin preparations may surprisingly be lowered to more suitable levels (e.g., below 1 μg/ml) by diafiltering purified toxin A and/or purified toxin B into 25 mM Tris (e.g., to remove MgCl₂) and then into a phosphate buffer (e.g., 100 mM PO₄, pH 7) using, for instance, tangential flow filtration (e.g., with flat stock Millipore PES50K) (e.g., as opposed to hollow-fiber or other type of membrane). The resulting lower concentration of TRIS may, in some embodiments, allow one to more effectively adjust the amount of formaldehyde required to effect the toxoiding process. Other embodiments may involve, for instance, using buffers that do not contain amine groups (e.g., MEM, acetate, citrate) and/or a pH-controlled aqueous solution (e.g., saline or water to which acid or base may be added).

Thus, in some preferred embodiments, in the toxoiding reactions, Tris may be replaced by another buffer such as a phosphate buffer. For instance, as described in the Examples, clarified C. difficile culture filtrate may be processed (e.g., concentrated and diafiltered such as by tangential flow filtration) into a Tris buffer (e.g., 50 mM Tris/NaCl/0.2 mM EDTA/1 mM DTT, pH 7.5). The resulting solution may then be filtered (e.g., using a membrane filter), ammonium sulfate concentration adjusted to about an appropriate amount (e.g., to about 0.4M) and then a further filtration may be performed (e.g., using a membrane filter). This aqueous solution, containing C. difficile toxin A and toxin B, may then be subjected to hydrophobic interaction chromatography and the toxins bound to a size exclusion (e.g., sepharose) column that may be washed with a Tris buffer. The C. difficile toxins may then be eluted with a Tris buffer containing DTT and IPA, pooled and adjusted to a conductivity of about 9 mS or less using WFI. These C. difficile toxins (in pooled elutate) may then be further purified by another method such as anion exchange chromatography involving the equilibration with a Tris buffer. Toxin A may then be eluted with a low-salt Tris buffer and toxin B with a high salt Tris buffer. The solutions containing purified toxin A or purified toxin B may each then be concentrated and diafiltered into a phosphate buffer such as 100 mM PO₄, pH 7 (where the residual TRIS values are preferably below about 1 to about 5 μg/ml). It has been found that lower concentrations of phosphate (e.g., 20 mM) may not be appropriate and may lead to increased multimerization (which should be minimized where possible). Thus, preferred suitable phosphate buffers may include any concentration of phosphate from above about 20 mM up to about 200 mM such as, for instance, about any of 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mM. As shown in the Examples herein, then, Toxin A may be converted to Toxoid A by mixing Toxin A with a formulation comprising about 0.21% (w/v) formaldehyde in 100 mM PO₄, pH 7 at about 25° C. for about six days. And in some preferred embodiments, as shown in the Examples herein, Toxin B may be converted to Toxoid B by mixing Toxin B with a formulation of about 0.41% (w/v) formaldehyde in 100 mM PO₄, pH 7 at about 25° C. for about 13 days. Other suitable buffers are also contemplated as would be understood by those of ordinary skill in the art.

One of ordinary skill in the art may determine whether a particular condition (e.g., buffer (or component thereof), time, temperature) is suitable for use in preparing and/or maintaining Toxoid A and/or Toxoid B compositions by assaying the same to determine whether the characteristics of the compositions are acceptable. For instance, the compositions may be tested using a cytotoxicity assay (e.g., using the IMR-90 cell line (see, e.g., the Examples) or Vero cells), anion exchange high-performance liquid chromatography (AEX-HPLC), size exclusion high-performance liquid chromatography (SEC-HPLC), enzyme-linked immunosorbent assay (ELISA), concentration measured using absorbance at 280 nm, reversion analysis (see, e.g., the Examples), and/or in vivo potency assay (e.g., hamster potency assay as described in the Examples). Compositions prepared under favorable conditions may typically exhibit any one or more of: little to no cytotoxicity for the cells monitored in cytotoxicity assays; AEX-HPLC and/or SEC-HPLC chromatograms showing little to no (or at least less under one condition versus another, less being preferable) multimerization of the toxoid(s); an ELISA/A280 value closer to 1 (e.g., as compared to compositions prepared under unfavorable conditions that may typically exhibit ELISA/A280 values further from 1); little to no reversion from toxoid to toxin during the testing period; and/or immunogenicity during in vivo assays (e.g., a Log10 titer of 4.8 or higher in a hamster potency assay). Other methods may also be used to make these determinations as may be determined by those of ordinary skill in the art.

The methods described herein are applicable to toxins from virtually any strain of C. difficile. Preferred strains of C. difficile are strains which produce Toxin A and/or B and include for example, but are not limited to strains of toxinotype 0 (e.g., VPI10463/ATCC43255, 630), III (e.g., 027/NAP/B1), V (e.g., 078) and VIII (e.g., 017). Methods are also applicable to C. difficile toxins produced using recombinant methods. The toxins (e.g., Toxin A and/or Toxin B) may be purified from culture filtrates of C. difficile using methods known in the art (e.g., U.S. Pat. No. 6,669,520). Exemplary methods of purifying toxins from culture filtrates of C. difficile are described in the Examples herein. Preferably the toxins have a purity of about any of 75%, 80%, 85%, 90%, 95%, 99% or more. The toxins may be inactivated together or separately. For example, the purified toxins may be mixed at a desired Toxin A: Toxin B ratio (e.g., 3:1, 3:2, 5:1, 1:5) and then inactivated or may be inactivated individually. Preferably the toxins are individually inactivated to produce toxoids. The term “toxoid” is used herein to describe a toxin that has been partially or completely inactivated by chemical treatment. A toxin is said to be inactivated if it has less toxicity (e.g., 100%, 99%, 95%, 90%, 80%, 75%, 60%, 55%, 50%, 25% or 10% or less toxicity) than untreated toxin, as measured, for example, by an in vitro cytotoxicity assay or by an in vivo assay. As disclosed herein, the toxins are inactivated using formaldehyde treatment. Other possible chemical means include for example, glutaraldehyde, peroxide, β-priopiolactone or oxygen treatment.

Inactivation may be carried out by incubating the toxin(s) with an amount of formaldehyde that prevents reversion of a toxoid into a toxin. Reversion may be prevented by including in a buffer comprising purified Toxin A or Toxin B a suitable amount of formaldehyde. The amount of formaldehyde in the buffer may be adjusted to maintain an appropriate concentration of formaldehyde to prevent reversion. To this end, a residual concentration of formaldehyde may be included in the buffer (and/or pharmaceutical composition). A residual concentration of formaldehyde is one that prevents reversion and/or presents a low risk of side effects to one to whom a composition described herein is administered. For instance, a residual formaldehyde concentration may range from about any of 0.0001% to 0.025% formaldehyde (w/v) (e.g., about any of 0.004%, 0.008%, 0.016%, or about 0.01%), about 0.001% to about 0.020% (w/v), about 0.004% to about 0.020% (w/v) (e.g., about 0.016%±0.04%), or about 0.004% to 0.010% (w/v) (e.g., about 0.008%), among other ranges. Prevention of reversion is typically found where no detectable cytotoxicity is observed following storage at 37° C. by in vitro assay such as for example, by the in vitro assay described herein (see, e.g., the cytotoxicity assays in the Examples). “Substantial” prevention of reversion typically means that 10% or less of the toxoid reverts into toxin following storage at 37° C. by the in vitro assay described in the Examples. A suitable in vitro cytotoxicity assay may be the cell-based florescence assay using, for instance, Vero cells. Another suitable in vitro cytotoxicity assay may be performed using IMR90 cells (e.g., ATCC® Accession No. CCL-186). Toxicity of the test material (e.g., toxoid) may be determined as the minimum concentration at which 50% of the cells become rounded as compared to their normal striated morphology (e.g., the MC-50). As described in the Examples herein, vaccine compositions comprising toxoids made by the methods described herein and formaldehyde of 0.008% or less showed no detectable cytotoxicity following storage at 37° C. by in vitro assay. Physicochemical analysis (e.g., anion exchange chromatography) may also be used to ascertain reversion but the in vitro cytotoxicity assay may be more informative. The potency of the toxoids may also be measured by a hamster in vivo potency assay which measures the mean of log10 anti-Toxin A or anti-Toxin B IgG titer.

In some embodiments, the appropriate amount of formaldehyde may be added to the toxins from a solution of 37% formaldehyde. The toxins are preferably in a suitable buffer solution (e.g., 100 mM sodium phosphate buffer, pH 7.0) prior to the addition of formaldehyde. Toxin concentration therein may be, for example, about 0.1 to about 5 mg/mL (e.g., 0.5 mg/mL). To begin the inactivation process, the toxins may initially be mixed with suitable concentration of formaldehyde (e.g., from about 0.1% to about 0.6%) for a suitable period of time (e.g., ten minutes). For example, purified Toxin A (0.5 mg/ml purified Toxin A in 100 mM sodium phosphate, pH 7.0) may be mixed in about 0.2% formaldehyde for about ten minutes. And purified Toxin B (e.g., 0.5 mg/ml purified Toxin B in 100 mM sodium phosphate, pH 7.0) may be mixed in about 0.4% formaldehyde for about ten minutes. Such mixtures may then be filtered (e.g., using 0.2 μm membrane filer) to remove small protein aggregates that may affect the protein concentration by adsorbance at 280 nm (e.g., allowing for precise formulation of the pharmaceutical composition at the intended Toxoid A:Toxoid B ratio). Inactivation may then be continued by incubating the mixture for about one to about 21 days (e.g., about two days, about six days, or about 13 days). For instance, the Toxin A mixture may be incubated in 13 days or less (e.g., about two days, about six days or about 13 days) at a suitable temperature (e.g., about 25° C.). The Toxin B mixture may be incubated for 21 days or less (e.g., about two days, about six days, or about 13 days) at a suitable temperature (e.g., about 25° C.). In this way, preparations of Toxoid A and/or Toxoid B may be provided. Such preparations typically comprise at least about any of 90%, 95%, 99% or 100% toxoid (e.g., inactivated toxin).

Although these toxoid preparations may be mixed directly with buffer, preferably the preparations are concentrated and diafiltered into an appropriate buffer solution. Preferably, concentration and diafiltration is done using tangential flow filtration to minimize protein shear while ensuring removal of formaldehyde and exchange into buffer. The buffer preferably includes at least one or more pharmaceutically acceptable excipients that increase the stability of the toxoids and/or delay or decrease aggregation of the toxoids. Excipients suitable for use include for example but are not limited to sugars (e.g., sucrose, trehalose) or sugar alcohols (e.g., sorbitol), and salts (sodium chloride, potassium chloride, magnesium chloride, magnesium acetate) or combinations thereof. Additionally, suitable excipients may be any of those described in, for example, US Pat. Pub. 2011/045025 (Ser. No. 12/667,864). Following inactivation, the solutions of inactivated toxins (i.e., toxoids) may be concentrated and/or ultrafiltered and/or diafiltered and stored in an appropriate buffer (such as, for example, but not limited to, about 5 to about 100 mM (e.g., about any of 5, 10, 15, 20, 25, 30, 35, 40, 45, 50, 55, 60, 65, 70, 75, 80, 85, 90, 95, or 100 mM citrate, phosphate, glycine, carbonate, bicarbonate, or the like, buffer) at a pH 8.0 or less (e.g., 6.5-7.7 such as about any of 6.5, 6.6, 6.7, 6.8, 6.9, 7.0, 7.1, 7.2, 7.3, 7.4, 7.5, 7.6, 7.7, 7.8, 7.9 or 8.0) (e.g., 20 mM citrate, pH 7.5) that prevents, or substantially prevents, reversion of the toxoids into a cytotoxic form (e.g., into a toxin). An exemplary buffer may be, for instance, 20 mM citrate, pH 7.5, 5%-8% sucrose, containing a suitable amount of formaldehyde (e.g., 0.016% (w/v)). Other buffers and the like may also be suitable, as would be understood by those of ordinary skill in the art.

The toxoids may be formulated for use as pharmaceutical compositions (e.g., immunogenic and/or vaccine compositions). For example, compositions comprising the C. difficile toxoids can be prepared for administration by suspension of the toxoids in a pharmaceutically acceptable diluent (e.g., physiological saline) or by association of the toxoids with a pharmaceutically acceptable carrier. Such pharmaceutical formulations may include one or more excipients (e.g., diluents, thickeners, buffers, preservatives, adjuvants, detergents and/or immunostimulants) which are known in the art. Suitable exicipents will be compatible with the toxoid and with the adjuvant (in adjuvanted compositions), with examples thereof being known and available to those of ordinary skill in the art. Compositions may be in liquid form, or lyophilized (as per standard methods) or foam dried (as described, e.g., in U.S. Pat. Pub. 2009/110699). An exemplary lyophilized vaccine composition may comprise for example, Toxoids A and B, 20 mM citrate, 8% sucrose, 0.016% formaldehyde, pH 7.5.

To prepare a vaccine for administration, a dried composition may be reconstituted with an aqueous solution such as, for example, water for injection, or a suitable diluent or buffer solution. In certain examples, the diluent includes formaldehyde as described herein. The diluent may include adjuvant (e.g., aluminum hydroxide) with or without formaldehyde. An exemplary diluent may be an aqueous solution of NaCl and aluminum hydroxide. Such a diluent may be used to reconstitute the dried composition. The pharmaceutical compositions may comprise a dose of the toxoids of about 10 to 150 μg/mL (e.g., any of about 10, 20, 30, 40, 50, 60, 70, 80, 90, 100, 110, 120, 130, 140 or 150 μg/mL). Typically, a volume of a dose for injection is about 0.5 mL or 1.0 mL. Dosages can be increased or decreased as to modulate immune response to be induced in a subject. The toxoids can be administered in the presence or absence of an adjuvant, in amounts that can be determined by one skilled in the art. Adjuvants of use include aluminum compounds, such as aluminum hydroxide, aluminum phosphate and aluminum hydroxyl phosphate.

The immunological and/or vaccine compositions can be administered by the percutaneous (e.g., intramuscular, intravenous, intraperitoneal or subcutaneous), transdermal, mucosal route in amounts and in regimens determined to be appropriate by those skilled in the art to subjects that have, or are at risk of developing, symptomatic C. difficile infection. These subject populations include, for example, subjects that have received broad spectrum antibiotics, such as hospitalized elderly patients, nursing home residents, chronically ill patients, cancer patients, AIDS patients, patients in intensive care units, and patients receiving dialysis treatment. The vaccine can be administered 1, 2, 3, 4 or more times. When multiple doses are administered, the doses can be separated from one another by, for example, one week, one month or several months. Thus, this disclosure also provides methods of eliciting an immune response against the toxins, toxoids, and/or C. difficile by administering the pharmaceutical compositions to a subject. This may be achieved by administration of the pharmaceutical compositions (e.g., immunogenic compositions and/or vaccines) described herein to the subject to effect exposure of the toxoids to the immune system of the subject. Thus, the immunogenic compositions and/or vaccines may be used to prevent and/or treat symptomatic C. difficile infections.

Compositions may be included in a kit (e.g., a vaccine kit). For example, the kit may comprise a first container containing a composition described herein in dried form and a second container containing an aqueous solution for reconstituting the composition. The kit may optionally include the device for administration of the reconstituted liquid form of the composition (e.g., hypodermic syringe, microneedle array) and/or instructions for use. Such kits are possible since it has been found that compositions as described herein can have good stability and remain non-cytotoxic following storage periods at moderate temperatures (e.g., at about 2-8° C.) and higher temperatures (e.g., at about 15° C., 25° C., 37° C. or higher). In certain examples, as described further below, compositions remained non-cytotoxic (e.g., without evidence of reversion) following storage at 37° C.

Thus, this disclosure provides methods for producing C. difficile toxoids by, for instance, inactivating purified C. difficile Toxin A and/or purified C. difficile Toxin B by incubation with about 0.15%-0.5% formaldehyde (w/v) at about 17-32° C. for about two to about 21 days. In some embodiments, Toxin A may be incubated with about 0.2% formaldehyde at about 25° C. for about two days to produce Toxoid A. In some embodiments, Toxin B is incubated with about 0.4% formaldehyde at about 25° C. for about 13 days to produce Toxoid B. Compositions comprising Toxoid A and/or Toxoid B prepared by such methods are also provided. Methods are also provided for preparing immunogenic compositions comprising purified C. difficile Toxoid A and purified C. difficile Toxoid B by combining purified C. difficile Toxoid A and purified C. difficile Toxoid B with a composition comprising a residual amount of formaldehyde (e.g., about any of 0.001% to 0.025%, such as about any of 0.004%, 0.008%, or 0.016% (w/v)). In some embodiments, the methods may provide compositions of C. difficile Toxoid A and/or purified C. difficile Toxoid B that are stable at 37° C. for up to about six weeks. Thus, in some embodiments, the methods described herein may also comprise inactivating purified C. difficile Toxin A or purified C. difficile Toxin B by incubation with about 0.15%-0.5% formaldehyde (w/v) at about 17-32° C. for about two to about 21 days; and, combining C. difficile Toxoid A and purified C. difficile Toxoid B with a composition comprising a residual amount of formaldehyde. The C. difficile Toxoids A and B compositions prepared by such methods may be stable at 37° C. for up to about six weeks. The residual amount of formaldehyde in such compositions may be about any of 0.001% to 0.025%, 0.004%, 0.008%, or 0.016% (w/v). The composition may also comprise about 20 mM citrate, pH 7.5, 4% to 8% sucrose, and 0.016% formaldehyde. In some embodiments, the composition may be lyophilized. These methods may also comprise providing a C. difficile culture comprising Toxin A and Toxin B and purifying the Toxin A and Toxin B from the culture. C. difficile Toxoids A or B produced in accordance with these method are also provided. In some embodiments, such compositions are vaccines (e.g., compositions that provide a protective, prophylactic, and/or therapeutic response against symptomatic C. difficile infection). The compositions (e.g., vaccine compositions) may comprise Toxoid A and Toxoid B in an A:B ratio of 5:1 to 1:5 such as e.g., 3:1 or 3:2. In some embodiments, the composition may be lyophilized, freeze dried, spray dried, or foam dried, or in liquid form. Such compositions may comprise one or more pharmaceutically acceptable excipients. The compositions may include a buffer such as for example, a citrate, phosphate, glycine, carbonate, or bicarbonate buffer, or a pH-controlled aqueous solution, and/or one or more sugars (e.g., sucrose, trehalose) and/or sugar alcohol (sorbitol). Other embodiments will be apparent to those of ordinary skill in the art.

A “purified” toxin typically means that the toxin has been isolated, for example, from culture filtrate and purified at least to some extent using methods known in the art. Exemplary methods of purifying toxins are described herein, for example. In some embodiments, a purified toxin may have a purity of about any of 75%, 80%, 85%, 90%, 95%, 99% or more. Similarly, a “purified” toxoid may be a toxoid that has a purity of about any of 75%, 80%, 85%, 90%, 95%, 99% or more.

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. For instance, the terms “about” or “approximately” may include values +/−10% of the indicated value (e.g., “about 30° C.” may mean any value between 27° C. to 33° C., including but not limited to 30° C.

As used herein, a subject or a host is meant to be an individual. The subject can include domesticated animals, such as cats and dogs, livestock (e.g., cattle, horses, pigs, sheep, and goats), laboratory animals (e.g., mice, rabbits, rats, guinea pigs) and birds. In one aspect, the subject is a mammal such as a primate or a human.

The terms “incubating”, “mixing” and “storing” (or synonyms and/or derivatives thereof) may be used interchangeably. For instance, a toxin may be incubated with a solution comprising formaldehye. Such an incubation may optionally mean, for instance, that the composition is being actively combined by motion (e.g., using a mixing bar of the like) or is being maintained in essentially a stationary state.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

When the terms prevent, preventing, and prevention are used herein in connection with a given treatment for a given condition (e.g., preventing symptomatic infection), it is meant to convey that the treated subject either does not develop a clinically observable level of the condition at all, or develops it more slowly and/or to a lesser degree than he/she would have absent the treatment. These terms are not limited solely to a situation in which the subject experiences no aspect of the condition whatsoever. For example, a treatment will be said to have prevented the condition if it is given during exposure of a subject to a stimulus that would have been expected to produce a given manifestation of the condition, and results in the subject's experiencing fewer and/or milder symptoms of the condition than otherwise expected. A treatment can “prevent” symptomatic infection by resulting in the subject displaying only mild overt symptoms of the infection; it does not imply that there must have been no C. difficile microorganism present.

Similarly, reduce, reducing, and reduction as used herein in connection with the risk of infection with a given treatment (e.g., reducing the risk of a symptomatic C. difficile infection) typically refers to a subject developing an infection more slowly or to a lesser degree as compared to a control or basal level of developing an infection in the absence of a treatment (e.g., administration or vaccination using toxoids disclosed). A reduction in the risk of symptomatic infection may result in the subject displaying only mild overt symptoms of the infection or delayed symptoms of infection; it does not imply that there must have been no C. difficile microorganism present.

All references cited within this disclosure are hereby incorporated by reference in their entirety. Certain embodiments are further described in the following examples. These embodiments are provided as examples only and are not intended to limit the scope of the claims in any way.

### EXAMPLES

The following examples are provided solely for purposes of illustration and are not intended to limit the scope of the disclosure. Changes in form and substitution of equivalents are contemplated as circumstances may suggest or render expedient. Although specific terms have been employed herein, such terms are intended in a descriptive sense and not for purposes of limitations. Methods of molecular genetics, protein biochemistry, and immunology used, but not explicitly described in this disclosure and these Examples, are amply reported in the scientific literature and are well within the ability of those skilled in the art.

### Example 1

A C. difficile working seed (strain VPI10463/ATCC43255) was used to inoculate preconditioned culture medium comprising soy peptone, yeast extract, phosphate buffer and sodium bicarbonate, pH 6.35-7.45 (SYS medium) and scaled up from a 4 mL Working Cell Bank (WCB) vial to a 160 L culture. Upon reaching the desired density and the 10-12 hour incubation period, the entire 160 L of culture was processed for clarification and 0.2 μm filtration. The culture from one more production fermentor was harvested and subjected to membrane filtration (e.g., using a Meisner membrane filter) to remove C. difficile cells and cell debris impurities. The resulting clarified culture filtrate was concentrated and diafiltered by tangential flow filtration into 50 mM Tris/NaCl/0.2 mM EDTA/1 mM DTT, pH 7.5. The resulting solution was filtered using a membrane filter, the concentration of ammonium sulfate was increased (e.g., to about 0.4M) and then a further filtration was performed (e.g., using a membrane filter). This aqueous solution contained C. difficile toxin A and toxin B. The aqueous solution was subjected to hydrophobic interaction chromatography. The C. difficile toxins were bound to a sepharose column. The column was washed with a Tris buffer and two fractions of the C. difficile toxins were eluted with a Tris buffer containing DTT and IPA. The two toxin fractions eluted from HIC were pooled and the conductivity adjusted to 9 mS or less using WFI. The C. difficile toxins (in pooled elutate) were further purified by anion exchange chromatography. The eluted aqueous solution was passed through an anion exchange column to bind toxins to column. The column was equilibrated with a Tris buffer and toxin A eluted with a low-salt Tris buffer and toxin B was eluted with high salt Tris buffer. Purified toxin A and purified toxin B were each concentrated and diafiltered into 100 mM PO₄, pH 7. Protein concentration was about 0.5 mg/mL and purity of each toxin was 90% or greater.

A 37% formaldehyde solution was added aseptically to each of the Toxin A diafiltrate and the Toxin B diafiltrate to obtain a final concentration of 0.42%. The solutions were mixed and then stored at 2-8° C. for 18-22 days. Following inactivation, the toxin diafiltrates were dialyzed into formulation buffer (20 mM citrate/5% sucrose, pH 7.5). The formaldehyde concentration was adjusted as required by adding 37% formaldehyde solution. Toxoids A and B were combined in a ratio of 3:2 (A:B) by weight and lyophilized. The lyophilized product comprised Toxoid A (0.24 mg/mL), Toxoid B (0.16 mg/mL), 20 mM sodium citrate, 5% (w/v) sucrose and the indicated concentration of formaldehyde.

A reversion analysis was performed to observe the potential reversion over a 6 week period at 37° C. Compositions comprising Toxoid A and Toxoid B were formulated with differing amounts of residual formaldehyde (0%, 0.008%, and 0.016% (w/v)), stored at either 37° C. or 4° C., and tested via cytotoxicity assay weekly for 6 weeks. Data from these studies are set out in Table 1. At 4° C., the product passes reversion analysis even with no residual formaldehyde added. However, at 37° C., 0.016% residual formaldehyde is needed to pass the reversion test.

TABLE 1
Reversion Analysis of Drug Product Stored at 37° C.

| | Day 7 | Day 14 | Day 21 | Day 28 | Day 35 | Day 42 |
|---|---|---|---|---|---|---|
| 4° C. | | | | | | |
| 0% | − | − | − | − | − | − |
| 0.008% | − | − | − | − | − | − |
| 0.016% | − | − | − | − | − | − |
| 37° C. | | | | | | |
| 0% | + | + | + | + | + | + |
| 0.008% | − | + | + | − | − | − |
| 0.016% | − | − | − | − | − | − |

* − = no cytotoxicity detected; + = cytotoxic

### Example 2

The experiments described herein were performed to identify a toxoiding method that would provide toxoids stable at 37° C. A C. difficile working seed (strain VPI10463/ATCC43255) was used to inoculate preconditioned culture medium (comprising soy peptone, yeast extract, phosphate buffer and D-sorbitol, pH 7.1-7.3) in a sterile disposable bag and culture was incubated at 35-39° C. until target OD was achieved. The 30 L Seed 1 culture was used to inoculate culture medium in a 250 L sterile disposable culture bag and culture was incubated at 35-39° C. until target OD is achieved. The Seed 2 culture was used to inoculate 1000 L sterile disposable culture bags and culture was incubated at 35-39° C. until target OD is achieved. The culture from one more production fermentor was harvested and subjected to depth filtration (e.g., using a Pall Depth 700 p/80 p/0.2 um 0.02 msq/L) to remove C. difficile cells and cell debris impurities and simultaneously cooled (e.g., about 37° C.-19° C.) to limit protease activity. The resulting clarified culture filtrate was concentrated and diafiltered by tangential flow filtration using flat stock Millipore and at a temperature of about 4° C. (for reduced protease activity) into 25 mM Tris/50 mM NaCl/0.2 mM EDTA, pH 7.5-8.0 and with no added DTT. The resulting solution was filtered using a membrane filter, the concentration of ammonium sulfate was increased (e.g., to about 0.9M) and then a further filtration was performed (e.g., using a membrane filter). This aqueous solution contained C. difficile toxin A and toxin B. The aqueous solution was subjected to hydrophobic interaction chromatography. The C. difficile toxins were bound to a butyl Sepharose resin (such as e.g., GE Butyl S FF Sepharose). The column was washed with 0.9 mM ammonium sulphate 25 mM Tris, pH 8.0 and C. difficile toxins were eluted with 25 mM Tris, pH 8.0 and conductivity adjusted to 7 mS or less using WFI. The C. difficile toxins (in elutate) were further purified by anion exchange chromatography. The eluted aqueous solution was passed through an anion exchange column (e.g., Tosoh Q 650 M) to bind toxins to column. The column was equilibrated with 25 mM Tris pH 7.5 and toxin A was eluted with 27 mM MgCl₂ in 25mM Tris, pH 8.0, and toxin B was eluted with 135 mM MgCl₂ in 25 mM Tris, pH 8.0. Purified toxin A and purified toxin B were each concentrated and first diafiltered into 25 mM Tris (e.g., to remove MgCl₂) and then into 100 mM PO₄, pH 7. Average yield of toxin A was about 0.021 g pure toxin/L fermentation (UV280) and purity as evaluated by SDS Page was about 97.2% on average. Average yield of toxin B was about 0.011 g pure toxin/L fermentation (UV280) and purity as evaluated by SDS Page was about 93.9% on average. The toxins generated from this process exhibit a purity of 90% or higher and also show a decrease in the matrix residuals (e.g., tris(hydroxymethyl)aminomethane (TRIS)) left behind from prior process steps. The residual TRIS values in the toxin matrix from the process substantially as described in Example 1 varied—100-800 μg/ml where as residual TRIS values in the toxin matrix from the purification process described in this example are below 1 μg/ml (i.e., below limit of detection). In regards to the toxoiding reaction with formaldehyde, TRIS has an amine group that can effectively compete with the protein for formaldehyde mediated modification, thereby lowering the effective formaldehyde concentration in the reaction mixture. Accordingly, data suggests that toxoiding kinetics for the toxoids made by this process are faster as compared to kinetics for the toxoids prepared by the process described in Example 1.

A study was performed on the toxoiding process with respect to temperature and formaldehyde concentration and analyzed as a function of the toxoiding incubation period. The objective was to develop a robust toxoiding process that provided a better safety profile and better reversion characteristics than the toxoids generated using the earlier process (as described in Example 1) while maintaining the same level of immunogenicity. Toxoiding conditions that would yield a drug product that passes reversion analysis at 37° C. with the least amount of residual formaldehyde was desired. In these experiments, toxin concentrations were fixed at 0.5 mg/ml and all of the reactions were performed in 100 mM sodium phosphate buffer, pH 7.0. The temperatures evaluated for each of toxoiding reactions were 4° C., 15° C. and 25° C. Formaldehyde concentration varied between 0.21% (“0.2%”) or 0.42% (“0.4%”) for toxoid A reactions and varied between 0.42% (“0.4%”) and 0.84% (“0.8%”) for toxoid B reactions. For each of the reaction conditions, toxin concentrations were adjusted to 0.5 mg/ml and were performed at the 100 ml scale. Thirty-seven percent (37%) formaldehyde was then added to reach the targeted concentrations for each of the individual reactions. The reactions were gently stirred for 5-10 minutes and placed in incubators at the targeted temperatures (target temp achieved within 1 hour of incubation). Each of the individual reactions were monitored daily for a period up to 21 days. Samples were pulled and analyzed by cytotoxicity analysis, AEX-HPLC, SEC-HPLC, SDS-PAGE and TNBS assay. At certain time intervals depending on toxoiding conditions, samples were pulled, formulated and animal studies, reversion analysis and ELISA testing was performed.

### Kinetic Cytotoxicity Analysis

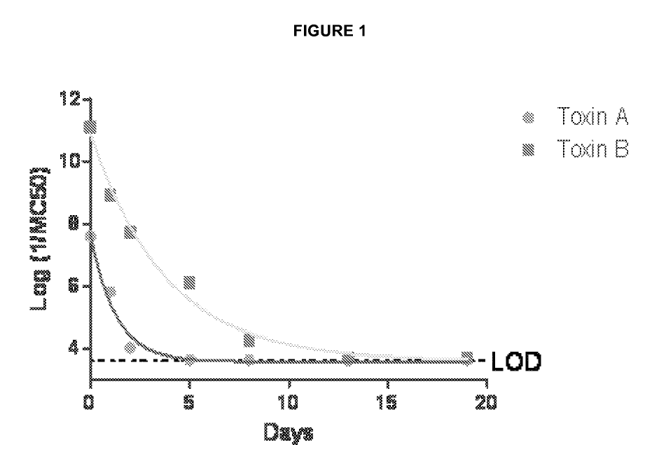

The toxoiding reaction was followed by cytotoxicity analysis and accordingly samples were pulled daily directly from the reaction mixture and submitted for same day analysis. The toxoiding process was followed by cytotoxicity on IMR90 cells and the kinetics of toxoiding was monophasic with Toxin A taking an average of 5±1 days for cytotoxicity neutralization and Toxin B taking close to 13±2 days (falling short of a 3 fold safety margin for the entire reaction). The data obtained using one batch is shown in FIG. 1. The y-axis contains MC50 values which is a reflection of the toxicity of the material and represents the minimum concentration at which the 50% of the cells become rounded in the presence of toxic material instead of their normal striated morphology. The MC 50 values for the two toxins differed by a factor of 1000; B was more cytotoxic with its MC50 value in the low pg/ml range. The absolute MC50 values for the toxoids were unknown as there was no cytotoxicity when tested at the highest concentration of 200 μg/ml in these experiments. The total time period for the inactivation process was 18-21 days.

Data from the cytotoxicity analysis for the toxoiding reactions of Toxin A and Toxin B are shown in Table 2. It portrays the amount of time (in days) needed to show a loss of cytotoxicity for each of the separate reactions of formaldehyde with the toxin. A few general trends are apparent from the data for the toxoiding reactions for Toxins A and Toxins B. As formaldehyde concentration is increased, the time required to inactivate the toxins is decreased. Additionally, as the temperature is increased for the reactions, the time required to inactivate the toxins is also decreased. The data suggests that the rate of toxoiding is accelerated with either an increase in temperature or formaldehyde concentration. Many potential conditions are identified from the kinetic cytotoxicity analysis and data suggests that a 3x safety margin could be achieved by extrapolating the initial loss of cytotoxicity three-fold. For example, Toxin A detoxifies at two days with 0.2% formaldehyde at 25° C., thus, applying an appropriate safety margin would minimally involve continuing the reaction for six days. Based on the acceptance criteria for loss of cytotoxicity (based on the kinetic analysis), a variety of toxoiding reaction conditions meet expectations, and further evaluation using other analysis will narrow down conditions.

TABLE 2
Cytoxicity Results for Kinetic Study*

| | Day 0 | Day 1 | Day 2 | Day 3 | Day 4 | Day 5 | Day 6 | Day 7 | Day 9 |
|---|---|---|---|---|---|---|---|---|---|
| Toxoid A, | + | + | + | + | + | + | + | + | + |
| 0.2%, 4° C. | | | | | | | | | |
| Toxoid A, | + | + | + | − | − | − | − | − | − |
| 0.2%, 15° C. | | | | | | | | | |
| Toxoid A, | + | + | − | − | − | − | − | − | N.D. |
| 0.2%, 25° C. | | | | | | | | | |
| Toxoid A, | + | + | + | − | − | − | − | − | − |
| 0.4%, 4° C. | | | | | | | | | |
| Toxoid A, | + | − | − | − | − | − | − | − | − |
| 0.4%, 15° C. | | | | | | | | | |
| Toxoid A, | + | − | − | − | − | − | − | − | N.D. |
| 0.4%, 25° C. | | | | | | | | | |
| Toxoid B, | + | + | + | + | + | + | + | + | − |
| 0.8%, 4° C. | | | | | | | | | |
| Toxoid B, | + | + | − | − | − | − | − | − | − |
| 0.8%, 15° C. | | | | | | | | | |
| Toxoid B, | + | − | − | − | − | − | − | − | − |
| 0.8%, 25° C. | | | | | | | | | |
| Toxoid B, | + | + | + | + | + | + | + | + | + |
| 0.4%, 4° C. | | | | | | | | | |
| Toxoid B, | + | + | + | + | + | − | − | − | − |
| 0.4%, 15° C. | | | | | | | | | |
| Toxoid B, | + | + | − | − | − | − | − | − | − |
| 0.4%, 25° C. | | | | | | | | | |

*+: Cytotoxic; −: No cytotoxicity detected; N.D.: not determined

### Kinetic AEX-HPLC Analysis of DoE Reactions

AEX-HPLC (extended gradient method) can be used as a tool to further evaluate the different toxoiding parameters. The AEX profile can be a valuable tool in narrowing down suitable toxoiding conditions. Two subpopulations are observed for both Toxoid A & Toxoid B in the AEX chromatogram both having longer retention times than the toxin. The populations of the peaks shift as the reaction progresses suggesting further modification to the toxin. Potentially, this reflects the formaldehyde reacting with amine groups on the toxin changing the charge characteristics on the protein to be less positive, thereby increasing the binding affinity with the column resin (quaternary ammonium resin). Temperature and Formaldehyde concentration can influence and “shift” the peak population profile as a function of time indicating more formaldehyde protein modification; for both Toxin A and Toxin B toxoiding reactions, a more rapid shift to the second peak population is observed with an increase temperature and formaldehyde concentration. From an evaluation standpoint, it would be more desirable to have a mono-dispersed profile at the second peak position to ensure more protein modification. For Toxoid A, conditions with 0.21% formaldehyde at 25° C., >6 days or 0.42% formaldehyde 15° C., >6 days gave the desired mono-dispersed 2nd peak profile. For Toxoid B, conditions with 0.4% or 0.8% formaldehyde at 15° C. for >10 days; or, 0.4% formaldehyde at 25° C. for >5 days resulted in the desired mono-dispersed 2nd peak profile. It is important to note that reactions with the highest formaldehyde concentrations and temperature began to produce more toxoid populations as a function of time suggesting more extensive protein modification (particularly in the case for the inactivation of toxin A at 0.4% formaldehyde, 25° C.).

### Kinetic SEC-HPLC Analysis

The SEC profile can be a valuable tool in narrowing down suitable toxoiding conditions. The chromatograms can give insights into the extent of multimerization that may occur as a result of formaldehyde induced intermolecular crosslinks. It is desired to minimize that amount of multimerization on the toxoids and achieve a profile similar to that with the product produced in Example 1. Individual reactions were monitored daily by SEC-MALS and qualitatively analyzed for the appearance of multimerization. All of the conditions analyzed for the Toxoid B reactions showed no multimerization. For Toxoid A excessive multimerization was observed mainly for the conditions with the highest formaldehyde concentration. Thus the SEC-MALS data does not discriminate for Toxoid B conditions with respect to temperature, time or formaldehyde concentration. However, the data suggests that higher temperature and formaldehyde concentration together can lead to multimerization for Toxoid A.

### Kinetic Amine Content (TNBS) Analysis

Formalin mediated toxoiding results in the reduction of free amine content on the protein (e.g., the ε-amino groups of lysine) through reaction to form formaldehyde based moieties. Attempts to monitor the extent of modification using a Trinitrobenzene sulfonic acid (TNBS) assay on the earlier material were made and the extent of modification at the end of the reaction was shown to be ˜35% and 65% for Toxoids A and B respectively (inverse of free amine content remaining). For this study, free amine content was also monitored using TNBS assay. The conditions show that as temperature and time are increased the % free amine content approaches an asymptote more rapidly. Thus the extent of amine modification can be maximally estimated ˜40% for Toxin A and 75% Toxin B (inverse of free amine content remaining). Although the amine content has little correlation with loss in cytotoxicity, it can be used to track the extent of reaction with formaldehyde and the toxins. For examples the amine modification appears to be complete with in 6 days with respect to A and ˜10 days with respect to B at 25° C. If the reaction is performed at lower temperatures, the time taken to achieve the same extent of amine modification increases. Thus data suggests that higher temperatures would lead to a more complete reaction in a shorter amount of time.

### Analysis of Antigenicity

An enzyme-linked immunosorbent assay (ELISA) can also be used as a tool to further evaluate the different toxoiding parameters. The ELISA profile of the product can be used to narrow down suitable toxoiding conditions. Toxoids generated were measured via ELISA against antibodies generated from the earlier material and analyzed as a function of toxoiding time. Here ELISA was used to detect the amount of toxin and compared against the concentration measured using absorbance at 280 nm. As the antigen progresses in the toxoiding reaction the ELISA value may drop off indicating a change from response observed with the Example 1 toxoids (potentially indicating multimerization). Although variability was noted in the assay, data suggests that higher temps and higher formaldehyde concentration lead to lower ELISA response. For example, the use of 0.4% formaldehyde at 25° C. results in ELISA values that fall faster than 0.2% formaldehyde at 25° C. Likewise, conditions with 0.4% formaldehyde, 25° C. results in ELISA values that fall faster than those at 0.4% formaldehyde at 4° C. As an evaluation tool, it was desired to keep the ELISA response above 70%; numerous conditions were identified.

### Analysis of Immunogenicity

Measurement of immunogenicity by hamster potency assay may be used to evaluate the toxoiding conditions. An IgG titer response of not less than 4.8 mean Log10 IgG titer response for Toxoid A and Toxoid B was selected. Toxoids generated from these studies were evaluated according to those specifications and further scrutinized as not having a significantly lower response from toxoids derived from the earlier conditions. Additionally, as all possible permeations (with respect to time, temperature and formaldehyde concentration) could not be evaluated, toxoids were selected based on kinetic cytotoxicity analysis (3x safety margin) as well as physiochemical characteristics described herein. The toxoids were formulated as bivalent material (non-lyophilized) for the hamster potency assay and the sera was analyzed for IgG response. All toxoiding conditions not only passed the potency specification (i.e., a mean IgG titer response of 4.8 Log10) but also had statistically equivalent titer response to the earlier (Example 1) material (no significant differences noted). Additionally, all of the sera was tested using an in vitro challenge assay and found to have neutralizing antibody activity. As a critical quality attribute, the data suggests that any of these toxoiding conditions could be acceptable.

### Reversion Analysis of Drug Product (“DP”)

Drug products (compositions comprising Toxoids A and B) were formulated using the Toxoids A and B prepared using the toxoiding conditions under evaluation. Formulations included either 0%, 0.004%, and in some cases 0.008% (w/v) residual formaldehyde. The formulations were prepared by removing all (or essentially all) of the formaldehyde from Toxoid A or B compositions and then spiking the cleared compositions with the indicated amounts of formaldehyde. The drug products were subjected to a reversion analysis conducted at 37° C. Data from the drug product reversion analysis is portrayed in Table 3. Drug products that tested negative for cytotoxicity are noted (−).

A number of drug product formulations passed the reversion analysis (i.e., had no detectable cytotoxicity following storage at 37° C.). Two drug products (with 0.004% or with 0.008% formaldehyde (“residual formaldehyde”)) had no detectable cytotoxicity following storage at 37° C.: (i) the drug product comprising Toxoid A inactivated by incubation 13 days, 0.2% formaldehyde, 15° C. and Toxoid B inactivated by incubation 13 days, 0.8% formaldehyde, 15° C. (Table 3, parameters of tests 13 and 14); and, (ii) the drug product comprising Toxoid A inactivated by incubation for 6 days 0.2% formaldehyde, 25° C., Toxoid B inactivated for 13 days 0.4% formaldehyde, at 25° C. (Table 3, parameters of tests 22 and 23). Several other drug products with 0.008% formaldehyde also had no detectable cytotoxicity following storage at 37° C. including for example, the drug product comprising Toxoid A inactivated by incubation for 13 days, 0.4% formaldehyde, 4° C. and Toxoid B inactivated for 21 days, 0.8%, 4° C. and the drug product comprising Toxoid A inactivated for 13 days, 0.4% formaldehyde, 4° C. and Toxoid B inactivated for 21 days, 0.8% formaldehyde and 4° C. Optimal toxoiding conditions identified from this analysis were: toxoiding of Toxin A: 0.5 mg/ml Toxin A, 0.21% formaldehyde, 25° C. in 100 mM NaPO₄ pH 7 for 6 days; and toxoiding of Toxin B: 0.5 mg/ml Toxin B, 0.42% formaldehyde, 25° C. in 100 mM NaPO₄ pH 7 for 13 days (Table 3, parameters of test 22). These conditions also had desirable profiles when measured by other physiochemical assays. AEX showed homogenous peak population for each toxoid, SEC MALS showed minimal multimerization and TNBS showed each reaction achieving maximal amine modification at the given time point. Additionally, the ELISA (A280) responses were maintained.

TABLE 3
Reversion Analysis (37° C.)

| | | | 37° C. | | | | | | |
|---|---|---|---|---|---|---|---|---|---|
| Test | Txd A | Txd B | Sample | Week 1 | Week 2 | Week 3 | Week 4 | Week 5 | Week 6 |
| 1 | 6 d, | 21 d, | DP + 0% Form. | +* | + | N.D. | N.D. | N.D. | N.D. |
| 2 | 0.4%, | 0.8%, | DP + 0.004% Form. | + | + | N.D. | N.D. | N.D. | N.D. |
| | 4° C. | 4° C. | | | | | | | |
| 3 | 13 d, | 21 d, | DP + 0% Form. | + | + | + | + | + | + |
| 4 | 0.4%, | 0.8%, | DP + 0.004% Form. | + | + | − | − | − | − |
| 5 | 4° C. | 4° C. | DP + 0.008% Form. | − | − | − | − | − | − |
| 6 | 6 d, | 13 d, | DP + 0% Form. | + | + | + | + | + | + |
| 7 | 0.2%, | 0.8%, | DP + 0.004% Form. | + | + | − | − | − | − |
| | 15° C. | 15° C. | | | | | | | |
| 8 | 6 d, | 13 d, | DP + 0% Form. | + | + | N.D. | N.D. | N.D. | N.D. |
| 9 | 0.2%, | 0.4%, | DP + 0.004% Form. | + | + | N.D. | N.D. | N.D. | N.D. |
| | 15° C. | 15° C. | | | | | | | |
| 10 | 6 d, | 18 d, | DP + 0% Form. | + | + | N.D. | N.D. | N.D. | N.D. |
| 11 | 0.2%, | 0.4%, | DP + 0.004% Form. | + | + | N.D. | N.D. | N.D. | N.D. |
| | 15° C. | 15° C. | | | | | | | |
| 12 | 13 d, | 13 d, | DP + 0% Form. | + | + | + | + | + | + |
| 13 | 0.2%, | 0.8%, | DP + 0.004% Form. | − | − | − | − | − | − |
| 14 | 15° C. | 15° C. | DP + 0.008% Form. | − | − | − | − | − | − |
| 15 | 13 d, | 13 d, | DP + 0% Form. | + | + | N.D. | N.D. | N.D. | N.D. |
| 16 | 0.2%, | 0.4%, | DP + 0.004% Form. | + | + | N.D. | N.D. | N.D. | N.D. |
| | 15° C. | 15° C. | | | | | | | |
| 17 | 13 d, | 18 d, | DP + 0% Form. | + | + | N.D. | N.D. | N.D. | N.D. |
| 18 | 0.2%, | 0.4%, | DP + 0.004% Form. | + | + | N.D. | N.D. | N.D. | N.D. |
| | 15° C. | 15° C. | | | | | | | |
| 19 | 6 d, | 6 d, | DP + 0% Form. | + | + | N.D. | N.D. | N.D. | N.D. |
| 20 | 0.2%, | 0.4%, | DP + 0.004% Form. | − | + | N.D. | N.D. | N.D. | N.D. |
| | 25° C. | 25° C. | | | | | | | |
| 21 | 6 d, | 13 d, | DP + 0% Form. | − | + | + | + | + | + |
| 22 | 0.2%, | 0.4%, | DP + 0.004% Form. | − | − | − | − | − | − |
| 23 | 25° C. | 25° C. | DP + 0.008% Form. | − | − | − | − | − | − |

Week 23 DP

| | | | | | | | | | |
|---|---|---|---|---|---|---|---|---|---|
| 24 | 6 d, | 21 d, | DP + 0% Form. | + | + | N.D. | N.D. | N.D. | N.D. |
| 25 | 0.4%, | 0.8%, | DP + 0.004% Form. | + | + | N.D. | N.D. | N.D. | N.D. |
| | 4° C. | 4° C. | | | | | | | |
| 26 | 13 d, | 21 d, | DP + 0% Form. | + | + | + | + | + | + |
| 27 | 0.4%, | 0.8%, | DP + 0.004% Form. | + | + | + | + | − | − |
| 28 | 4° C. | 4° C. | DP + 0.008% Form. | − | − | − | − | − | − |

DP = drug product;
Form. = formaldehyde;
+: Cytotoxic;
−: No cytotoxicity detected;
N.D.: not determined

Tables 1 and 3 indicate that the parameters 22 are optimal for preparing toxoids from Toxins A and B. These conditions are:

Preparation of toxoid A: 0.5 mg/ml Toxin A, 0.21% formaldehyde, 25° C. in 100 mM NaPO₄, pH 7 for six days; and,

Preparation of toxoid B: 0.5 mg/ml Toxin B, 0.42% formaldehyde, 25° C. in 100 mM NaPO₄, pH 7 for 13 days.

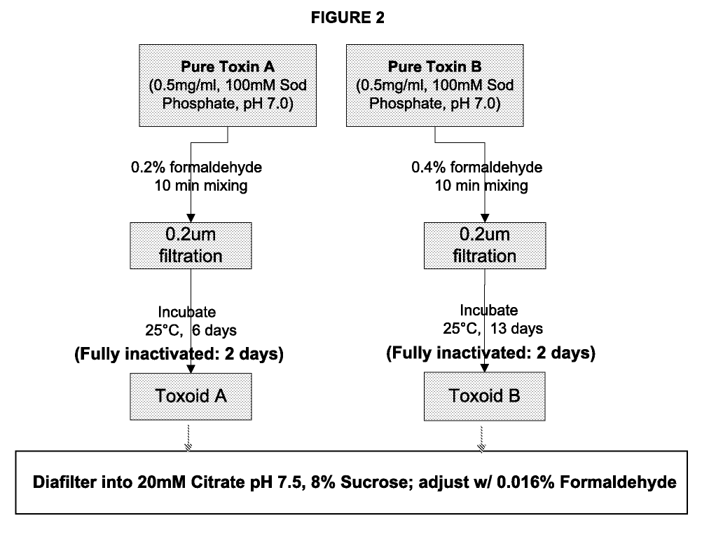

These procedures also included a ten minute mixing step followed by 0.2 μm filtration prior to the six day (Toxoid A) or 13 day (Toxoid B) incubation. Prior to testing for reversion at 37° C., Toxoid A and toxoid B were diafiltered into 20 mM citrate, pH 7.5, 0.004% formaldehyde. This procedure is illustrated in FIG. 2. It is also noted that 0.008% formaldehyde in the citrate buffer also typically provides good stability at 37° C.

This data is further confirmed by surprising immunological data (IgG response in hamsters) showing that longer incubation times resulted in lower ELISA values for Toxoid A, suggesting increased formaldehyde-induced toxin modification (ELISA/A280 at day 6=0.94; at day 12=0.64). In contrast, longer incubation times resulted in higher ELISA values for Toxoid B (ELISA/A280 at day 13=0.53; at day 20=0.73). Desirable ELISA/A280 values are those closer to 1.0. Those of ordinary skill in the art should understand that a 12 day incubation period for toxoiding Toxin A may be appropriate and a 20 day incubation period may be appropriate for toxoiding of Toxin B. However, even in view of this data, a 13 day incubation time was considered optimal for toxoiding Toxin B as described above.

### Scale Analysis

Toxoids were produced at a larger scale ( 1/10^{th} launch scale (200 L fermentation)) using the optimal toxoiding conditions identified; that is, Toxin A and Toxin B were inactivated using the following conditions: Toxoiding of A: 0.5 mg/mL toxin A, 0.21% (w/v) formaldehyde, 25° C. in 100 mM NaPO₄ pH 7 for 6 days; and, Toxoiding of B: 0.5 mg/mL toxin B, 0.42% (w/v) formaldehyde, 25° C. in 100 mM NaPO₄ pH 7 for 13 days. The kinetics of the toxoiding reaction was evaluated using toxoid samples taken at various time periods during the reaction. In comparison to the toxoids produced at small scale, the toxoids had an identical kinetic cytotoxicity profile, with a loss of cytotoxicity being observed on the 2^{nd} day of the reaction. In addition the toxoids had a similar AEX profile and similar amine modification (as measured by TNBS assay) to toxoids produced at small scale. The immunogenicity of the toxoids generated from the 1/10^{th} scale toxoiding reaction were also evaluated by the hamster potency assay. Like the toxoids produced at small scale, the toxoids gave a mean IgG titer response of 4.8 Log or higher and provided a titer response that was statistically equivalent to that of toxoids prepared in accordance to the process as set out in Example 1. Reversion analysis was conducted on drug product derived from 1/10^{th} scale toxoids and compared to drug product derived from identical toxoiding conditions at small scale. The drug product derived from toxoids at 1/10^{th} scale had the same reversion characteristics as those derived at the small scale and passed reversion even at 0.004% formaldehyde. Similar results were obtained with Toxoids produced at larger scales (e.g., using 1000 L and 2000 L fermentation cultures). The data from these studies show that the toxoiding method is scalable. The toxoids produced at large scale have identical kinetic cytotoxic profiles, hamster potency and reversion characteristics as those produced at small scale. In regards to reproducibility, the toxoiding process for Toxin A and Toxin B was reproduced more than 6 times and analysis showed similar lot to lot characteristics.

### Immunization Studies

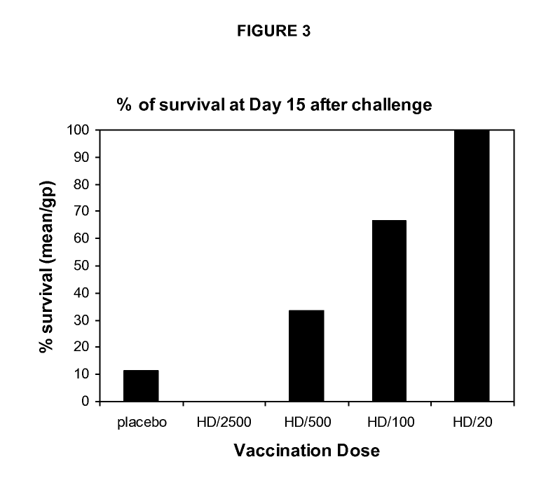

Purified C. difficile Toxoid A and C. difficile Toxoid B were prepared substantially in accordance with the methods described above (e.g., parameters 22 in Table 3) and formulated as vaccine compositions. Toxoids A and B were combined at a ratio of 3:2 by weight, formulated with a citrate buffer comprising sucrose (4.0% to 6.0% w/v) and formaldehyde (0.012% to 0.020% w/v) and lyophilized. Each composition was reconstituted with diluent as described below and adjuvanted with aluminum hydroxide prior to use as a vaccine. Syrian gold hamsters provide a stringent model for C. difficile vaccine development. After being pretreated with a single intraperitoneal (IP) dose of clindamycin antibiotic and after receiving an intragastric (IG) inoculation of toxigenic C. difficile organisms, the hamsters rapidly develop fulminant diarrhea and hemorrhagic cecitis and die within two to four days (e.g., without vaccination). The vaccine was reconstituted with diluent (comprising 0.57% sodium chloride and 800 μg/mL aluminum). The reconstituted vaccine contained 100 μg/dose toxoids, 0.008% formaldehyde and 400 μg/dose aluminum. Hamsters (9 hamsters/group) were vaccinated by three intramuscular immunizations (at Day 0, Day 14, and Day 27) with different doses of C. difficile vaccine (4 dilutions of human dose (100 μg/dose) (HD) or were injected with the placebo (AIOH). At Day 41, hamsters were pretreated with chemical form of Clindamycin-2-phosphate antibiotic at 10 mg/kg by IP route. At Day 42, after 28 hours pretreatment with antibiotic, hamsters were challenged by IG route with a lethal dose of spore preparation derived from C. difficile ATCC43255 strain. The protective efficacy was assessed by measuring the kinetics of apparition of symptoms associated with C. difficile infection and lethality. Results demonstrated that the vaccine protects hamsters against lethal challenge with C. difficile toxigenic bacteria in a dose-dependent manner, with 100% protection induced by vaccination with the dose HD/20 (5 μg Toxoid A+B in presence of 100 μg/mL AIOH) (FIG. 3). Immunized animals were protected against death and disease (weight loss and diarrhea). The results of this study are representative of several in vivo studies. Accordingly, toxoids prepared by the methods described herein provide protective immunity against C. difficile disease (symptomatic C. difficile infection).

While certain embodiments have been described in terms of the preferred embodiments, it is understood that variations and modifications will occur to those skilled in the art. Therefore, it is intended that the appended claims cover all such equivalent variations that come within the scope of the following claims.

1. A method of producing a C. difficile toxoid, the method comprising inactivating purified C. difficile Toxin A or purified C. difficile Toxin B by incubation with about 0.15%-0.5% formaldehyde (w/v) at about 17-32° C. for about two to about 21 days.

2. The method of claim 1 wherein Toxin A is incubated with about 0.2% formaldehyde at about 25° C. for about two days to produce Toxoid A.

3. The method of claim 1 wherein Toxin B is incubated with about 0.4% formaldehyde at about 25° C. for about 13 days to produce Toxoid B.

4. A composition comprising Toxoid A or Toxoid B prepared by the method of claim 1.

5. A method for preparing an immunogenic composition comprising purified C. difficile Toxoid A and purified C. difficile Toxoid B by combining purified C. difficile Toxoid A and purified C. difficile Toxoid B with a composition comprising a residual amount of formaldehyde.

6. The method of claim 5 wherein the purified C. difficile Toxoid A and purified C. difficile Toxoid B are stable at 37° C. for up to about six weeks.

7. The method of claim 5 wherein the residual amount of formaldehyde is about 0.004%, 0.008%, or 0.016% (w/v).

8. A composition prepared using a method of claim 5.

9. A method for preparing an immunogenic composition comprising purified C. difficile Toxoid A and purified C. difficile Toxoid B, the method comprising:
inactivating purified C. difficile Toxin A and purified C. difficile Toxin B by incubation with about 0.15%-0.5% formaldehyde (w/v) at about 17-32° C. for about two to about 21 days; and,
combining purified C. difficile Toxoid A and purified C. difficile Toxoid B with a composition comprising a residual amount of formaldehyde.

10. The method of claim 9 wherein the purified C. difficile Toxoid A and purified C. difficile Toxoid B are stable at 37° C. for up to about six weeks.

11. The method of claim 9 wherein the residual amount of formaldehyde is about 0.001%, 0.004%, 0.008%, or 0.016% (w/v).

12. The method of claim 1 wherein the Toxoid A and/or Toxoid B is maintained in a composition of 20 mM citrate, pH 7.5, 8% sucrose, and 0.016% formaldehyde.

13. The method of claim 1 wherein the composition comprising Toxoid A and/or Toxoid B is lyophilized.

14. A composition prepared using the method of claim 9.

15. The method of claim 1 further comprising providing a C. difficile culture comprising Toxin A and Toxin B and purifying the Toxin A and Toxin B from the culture.

16. C. difficile Toxoid A produced in accordance to the method of claim 1.

17. C. difficile Toxoid B produced in accordance to the method of claim 1.

18. A vaccine composition comprising C. difficile Toxoid A and C. difficile Toxoid B, wherein the C. difficile Toxoid A and Toxoid B are produced by the method of claim 1.

19. The vaccine composition of claim 18 wherein the vaccine composition comprises about 0.001% to 0.020% formaldehyde.

20. The vaccine composition of claim 19 wherein the vaccine composition comprises about 0.004% formaldehyde.

21. The vaccine composition of claim 19 wherein the vaccine composition comprises 0.008% formaldehyde.

22. The vaccine composition of claim 19 wherein the vaccine composition comprises about 0.016% formaldehyde.

23. A vaccine composition of claim 18 wherein the Toxoid A and the Toxoid B are present in the composition in a A:B ratio of 5:1 to 1:5.

24. A vaccine composition of claim 23 wherein the Toxoid A and the Toxoid B are present in the composition in a ratio of A:B of 3:1 or 3:2.

25. A vaccine composition of claim 18 wherein the vaccine composition is freeze dried, spray dried, or foam dried.

26. A vaccine composition of claim 18 wherein the vaccine composition is in liquid form.

27. A vaccine composition of claim 18 further comprising one or more pharmaceutically acceptable excipients.

28. A composition of claim 4 comprising a citrate, phosphate, glycine, carbonate, or bicarbonate buffer, or a pH-controlled aqueous solution.

29. The composition of claim 28 further comprising a sugar, or sugar alcohol.

30. The composition of claim 29 comprising sucrose and citrate.

31. A method for converting C. difficile Toxin A into a toxoid (Toxoid A) by incubating the Toxin A with about 0.21% (w/v) formaldehyde at about 25° C. for about six to 13 days.

32. The method of claim 31 wherein the incubating is for about six days.

33. The method of claim 31 wherein the incubating occurs in 0.21% (w/v) formaldehyde/100 mM PO₄, pH 7.

34. A method for converting C. difficile Toxin B into a toxoid (Toxoid B) by incubating the Toxin B with about 0.42% (w/v) formaldehyde at about 25° C. for about 13 to about 20 days.

35. The method of claim 34 wherein the incubating is for about 13 days.

36. The method of claim 34 wherein the incubating occurs in 0.42% (w/v) formaldehyde/100 mM PO₄, pH 7.
